(19) United States
(12) Patent Application Publication
(10) Pub. No.: US 2014/0276201 A1
(43) Pub. Date: Sep. 18, 2014

(54) SYSTEM AND METHOD FOR DETECTING TISSUE STATE AND INFECTION DURING ELECTROSURGICAL TREATMENT OF WOUND TISSUE

(71) Applicant: ARTHROCARE CORPORATION, AUSTIN, TX (US)

(72) Inventors: Jean Woloszko, Austin, TX (US); Thomas P. Ryan, Austin, TX (US); Kenneth R. Stalder, Redwood City, CA (US)

(73) Assignee: ARTHROCARE CORPORATION, AUSTIN, TX (US)

(21) Appl. No.: 14/202,400

(22) Filed: Mar. 10, 2014

Related U.S. Application Data
(60) Provisional application No. 61/788,706, filed on Mar. 15, 2013.

(51) Int. Cl. A61B 18/18; A61B 5/145

(52) U.S. Cl. CPC A61B 18/18; A61B 5/14546
USPC 600/562; 606/34; 606/33

(57) ABSTRACT

A method exposes a wound bed to electrosurgical treatment to generate fragmented wound tissue, gathers a molecular gaseous by-product sample of the fragmented wound tissue, and analyzes the molecular gaseous by-product sample of the fragmented wound tissue to generate a fragmented wound tissue compound analysis profile. The method further compares the fragmented wound tissue compound analysis profile with a database of known compound analysis profiles and provides a diagnosis of the wound tissue based on the comparison of compound analysis profiles.

### CROSS-REFERENCE TO RELATED APPLICATIONS

This application claims priority to U.S. provisional application No. 61/788,706, filed Mar. 15, 2013, entitled “SYSTEM AND METHOD FOR DETECTING TISSUE STATE AND INFECTION DURING ELECTROSURGICAL TREATMENT OF WOUND TISSUE”.

### FIELD OF INVENTION

This disclosure pertains to a detection system and method for determining the state of target tissue including the type of target tissue, the disease state of the target tissue, pathogen infection types and levels in the tissue, and biofilm presence to assist in the electrosurgical treatment of the target tissue, in particular, an electrosurgical treatment whereby an active electrode in the presence of plasma is directed to perforate and/or debride wound tissue, remove debris and pathogens from a wound bed, induce blood flow, and leverage the body's metabolic, vascular, molecular, and biochemical response to promote, stimulate, and stabilize the healing process.

### BACKGROUND OF THE INVENTION

Electrosurgical tissue treatment may be conducted on target tissue for a variety of reasons. The target tissue may be an organ or tissue structure requiring electrosurgical intervention or may be an infection field requiring surgical debridement or other electrosurgical intervention. Electrosurgical treatment may include removal of tumor tissue from organs or portions of the human body using energy-based surgical treatments such as laser ablation, cautery, plasma produced in liquid or gas plasma treatment applied to tumors as distinguished from the underlying organ or other tissue.

Similar treatment mechanisms may be applied to treatment of internal membranes such as those in otorhinolaryngological (ENT) applications. For example, sinuses may become infected severely enough to develop infection field biofilms that may be treated with electrosurgery. Ear, nose and throat infections are becoming more resistant to common treatment. This is due to the presence of biofilms which can be found in ear infections (with mucosal biofilms), as well as chronic sinusitis (also commonly related to biofilms). Biofilms have been demonstrated on tonsils, adenoids, and sinus locations and the biofilms interfere with the application of antibiotics. Electrosurgical removal of these biofilms in infected target treatment sites is advantageous for sterilization and promotion of healing.

Yet another example of electrosurgical treatment includes dermatological applications. One specific example of the use of electrosurgical treatment is treatment of chronic wounds. Wound healing is the body's natural response for repairing and regenerating dermal and epidermal tissue. Wound healing is generally categorized into four stages: 1) clotting/hemostasis stage; 2) inflammatory stage; 3) tissue cell proliferation stage; and 4) tissue cell remodeling stage. The wound healing process is complex and fragile and may be susceptible to interruption or failure, especially in the instance of chronic wounds. A wound that does not heal in a predictable amount of time and in the orderly set of stages for typical wound healing may be categorized as chronic.

Chronic wounds may become caught in one or more of the four stages of wound healing, such as remaining in the inflammatory stage for too long, and thereby preventing the wound healing process to naturally progress. Similarly, a chronic wound may fail to adequately finish one stage of healing before moving on to the next, resulting in interference between the healing stages and potentially causing processes to repeat without an effective end. By way of further example, during the stage of epithelialization in typical wound healing, epithelial cells are formed at the edges of the wound or in proximity to a border or rim surrounding the wound bed and proliferate over the wound bed to cover it, continuing until the cells from various sides meet in the middle. Affected by various growth factors, the epithelial cells proliferate over the wound bed, engulfing and eliminating debris and pathogens found in the wound bed such as dead or necrotic tissue and bacterial matter that would otherwise obstruct their path and delay or prevent wound healing and closure. However, the epithelialization process in chronic wounds may be short-circuited or ineffective as the epithelial cells, needing living tissue to migrate across the wound bed, do not rapidly proliferate over the wound bed, or in some instances do not adequately respond at all during this particular stage of wound healing. As such, a need arises with chronic wounds to sterilize the wound site, as well as to establish communication between healthy tissue and wound tissue to promote epithelialization, fibroblast and epithelial migration, and neovascularization, and to bridge the gaps (i.e., including but not limited to structural and vascular gaps) between vital tissue surrounding the wound bed and tissue on the periphery of and within the wound bed itself.

Certain chronic wounds can be classified as ulcers of some type (i.e., diabetic ulcers, venous ulcers, and pressure ulcers). An ulcer is a break in a skin or a mucus membrane evident by a loss of surface tissue, tissue disintegration, necrosis of epithelial tissue, nerve damage and pus. Venous ulcers typically occur in the legs and are thought to be attributable to either chronic venous insufficiency or a combination of arterial and venous insufficiency, resulting in improper blood flow and/or a restriction in blood flow that causes tissue damage leading to the wound. Pressure ulcers typically occur in people with limited mobility or paralysis, where the condition of the person inhibits movement of body parts that are commonly subjected to pressure. Pressure ulcers, commonly referred to as “bed sores,” are caused by ischemia that occurs when the pressure on the tissue is greater than the blood pressure in the capillaries at the wound site, thus restricting blood flow into the area.

For patients with long-standing diabetes and with poor glycemic control, a common condition is a diabetic foot ulcer, symptoms of which include slow healing surface lesions with peripheral neuropathy (which inhibits the perception of pain), arterial insufficiency, damage to small blood vessels, poor vascularization, ischemia of surrounding tissue, deformities, cellulitis tissue formation, high rates of infection and inflammation. Cellulitis tissue includes callous and fibrotic tissue. Thus, due to the often concomitant loss of sensation in the wound area, diabetic patients may not initially notice small, non-lesioned wounds to legs and feet, and may therefore fail to prevent infection or repeated injury. If left untreated a diabetic foot ulcer can become infected and gangrenous which can result in disfiguring scars, foot deformity, and/or amputation.

Example chronic wound beds 110 of a diabetic foot ulcer are illustrated in FIG. 1. A diabetic foot ulcer may develop on any position of the foot, and typically occur on areas of the foot subjected to pressure or injury and common areas such as: on the dorsal portion of the toes; the pad of the foot; and the heel. The wound tissue beds 110 shown in FIG. 1 may be examples of tissue treatment sites.

Typically, ulcer treatment is dependent upon its location, size, depth, and appearance to determine whether it is neuropathic, ischemic, or neuro-ischemic. Depending on the diagnosis, antibiotics may be administered and if further treatment is necessary, the symptomatic wound bed area is treated more aggressively (e.g., by surgical debridement using a scalpel, scissors, or other instrument to cut necrotic and/or infected tissue from the wound, mechanical debridement using the removal of dressing adhered to the wound tissue, or chemical debridement using certain enzymes and other compounds to dissolve wound tissue) to remove unhealthy wound tissue and induce blood flow and to expose healthy underlying structure. Often, extensive post-debridement treatment such as dressings, foams, hydrocolloids, genetically engineered platelet-derived growth factor becaplermin and bio-engineered skins and the like may also be utilized.

Additionally, several other types of wounds may progress to a chronic, non-healing condition. For example, surgical wounds at the site of incision may progress inappropriately to a chronic wound bed or may progress to pathological scarring such as a keloid scar. Trauma wounds may similarly progress to chronic wound status due to infection or involvement of other factors within the wound bed that inhibit proper healing. Burn treatment and related skin grafting procedures may also be compromised due to improper wound healing response and the presence of chronic wound formation characteristics. In various types of burns, ulcers, and amputation wounds, skin grafting may be required. In certain instances, patients with ischemia or poor vascularity may experience difficulty in the graft “taking” resulting in the need for multiple costly skin grafting procedures.

Various methods exist for treatment of chronic wounds, including antibiotic and antibacterial use, surgical or mechanical debridement, irrigation, topical chemical treatment, warming, oxygenation, and moist wound healing, which remain subject to several shortcomings in their efficacy. Electrosurgical treatment such as electrosurgical debridement provides added benefits, but is still fraught with some difficulty. Determining the level or type of infection or presence of biofilms and infection is difficult to assess during electrosurgical treatment. This is especially true because the electrosurgical treatment removes and alters the target tissue by volumetric dissociation of the target tissue, biofilms, and pathogens present in the wound bed. Progress of the electrosurgical treatment in removal of target tissue is similarly difficult to assess by a surgeon to ensure removal of only desired targeted tissue and not healthy tissue or tissue of a type that is different from the target tissue. Location of biofilms, infection, or tissue types within a patient space of the wound bed or treatment site is also difficult to assess during electrosurgical treatment. Post-debridement treatment may also depend on the state of the wound tissue after electrosurgical treatment. Tissue or pathogen analysis may take hours or days which is untenable during an ongoing electrosurgical treatment. Accordingly, there remains a need for new and improved systems and methods for use in detecting and determining the type and state of target tissue during the treatment of target tissue, such as wounds, that address certain of the forgoing difficulties.

### BRIEF DESCRIPTION OF THE DRAWINGS

It will be appreciated that for simplicity and clarity of illustration, elements illustrated in the Figures have not necessarily been drawn to scale. For example, the dimensions of some of the elements are exaggerated relative to other elements. Embodiments incorporating teachings of the present disclosure are shown and described with respect to the drawings presented herein, in which:

FIG. 1 is an illustration of ulcer locations on a foot;

FIG. 2 is an illustration of an electrosurgical system and compound analysis system adaptable for use with at least some of the embodiments of the present method;

FIG. 3 is an illustration of an electrode configuration for target tissue treatment and gaseous fluid gathering in accordance with at least some of the embodiments of the present method;

FIGS. 4A-D are illustrations of electrode configurations for target tissue treatment in accordance with at least some of the embodiments of the present method;

FIGS. 5A-D are illustrations of example portions of compound analysis profiles of tissue states in accordance with at least some of the embodiments of the present method;

FIG. 6 shows a method of target tissue analysis in accordance with at least some of the disclosed embodiments;

FIG. 7 shows another method of target tissue analysis in accordance with at least some of the disclosed embodiments;

FIG. 8 is an illustration of a diabetic foot ulcer on the pad of the foot with an embodiment of segmented wound bed location zones; and

FIG. 9 shows an algorithm in accordance with at least some of the embodiments of the present method.

### NOTATION AND NOMENCLATURE

Certain terms are used throughout the following description and claims to refer to particular system components. As one skilled in the art will appreciate, companies that design and manufacture electrosurgical systems may refer to a component by different names. Similarly, companies that develop and manufacture compound analysis systems may also refer to components by different names. This document does not intend to distinguish between components that differ in name but not function.

In the following discussion and in the claims, the terms “including” and “comprising” are used in an open-ended fashion, and thus should be interpreted to mean “including, but not limited to . . . . ” Also, the term “couple” or “couples” is intended to mean either an indirect or direct connection. Thus, if a first device couples to a second device, that connection may be through a direct connection or through an indirect electrical connection via other devices and connections.

Reference to a singular item includes the possibility that there are plural of the same items present. More specifically, as used herein and in the appended claims, the singular forms “a,” “an,” “said” and “the” include plural references unless the context clearly dictates otherwise. It is further noted that the claims may be drafted to exclude any optional element. As such, this statement serves as antecedent basis for use of such exclusive terminology as “solely,” “only” and the like in connection with the recitation of claim elements, or use of a “negative” limitation. Lastly, it is to be appreciated that unless defined otherwise, all technical and scientific terms used herein have the same meaning as commonly understood by one of ordinary skill in the art to which this invention belongs.

“Active electrode” shall mean an electrode of an electrosurgical wand which produces an electrically-induced tissue-altering effect when brought into contact with, or close proximity to, a tissue targeted for treatment, and/or an electrode having a voltage induced thereon by a voltage generator.

“Electrosurgical treatment” shall mean any energy-based volumetric dissociation of tissue by proximity of the energy-based treatment application whether plasma based, transmission based, thermal or cautery based, fluid jet systems and including treatment from monopolar or bipolar active electrodes or other instrument generating a plasma-based treatment in fluid such as Coblation® technology, gas-based plasma treatment of tissue, surgical laser ablation, other ablation due to energy application, and cauterization tools of any typical shape used in surgical applications.

“Fragmentation” shall mean volumetric alteration of tissue by application of treatment whether that is “electrosurgical treatment” or non-electrosurgical treatment with tissue treatment systems including, motorized or mechanical tissue treatment systems, or conventional treatment with scalpel, scissors, or other instruments to cut target tissue such as necrotic and/or infected tissue. Additional examples include treatment of target tissue such as wound tissue with mechanical debridement using the removal of dressing adhered to target tissue such as wound tissue or chemical treatment of target tissue using certain enzymes and other compounds to dissolve target tissue.

“Chronic wound tissue” shall mean wound tissue that does not heal in an orderly set of stages and in a predictable amount of time, including but not limited to wound tissue attributable to diabetic ulcers, venous ulcers, pressure ulcers, surgical wounds, trauma wounds, burns, amputation wounds, irradiated tissue, tissue affected by chemotherapy treatment, and/or infected tissue compromised by a weakened immune system, or any combination of the above.

“Physiological tissue types” shall mean any type of human tissue, diseased or healthy, that may be subject to electrosurgical treatment including but not limited to epidermal, dermal and sub-cutaneous layers of skin, other epithelial tissue, mucus membrane tissue, sinus tissue, connective tissues, fat tissues, musculo-skeletal tissues, cartilages, connector structure, membranes, brain and nervous system tissue, brain and nervous system membranes, ophthalmic, organ tissues such as liver, renal, prostate, uterine, pulmonary, tonsil, adenoid, bladder, gall bladder, gastro-intestinal, esophageal, spleen, reproductive, vascular and cardiac organ tissue, tumor tissue, infection site tissue infected by a variety of pathogens, and other tissues.

Where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

### DETAILED DESCRIPTION OF THE INVENTION

In the drawings and description that follows, like parts may be marked throughout the specification and drawings with the same reference numerals, respectively. The drawing figures are not necessarily to scale. Certain features of the invention may be shown exaggerated in scale or in somewhat schematic form and some details of conventional elements may not be shown in the interest of clarity and conciseness. The present invention is susceptible to embodiments of different forms. Specific embodiments are described in detail and are shown in the drawings, with the understanding that the present disclosure is to be considered an exemplification of the principles of the invention, and is not intended to limit the invention to that illustrated and described herein. It is to be fully recognized that the different teachings of the embodiments discussed below may be employed separately or in any suitable combination to produce desired results.

Electrosurgical apparatus and systems adaptable for use with the present method include any energy-based electrosurgical treatment of target tissue. Electrosurgical treatment and associated instrument systems are defined above and may include ablation due to energy transmission, generation of a plasma in a liquid, gaseous plasma generation, and thermal or cautery systems. Additionally volumetric dissociation via surgical fluid jets, such as VersaJet® water jet systems, are considered “electrosurgical treatment” for purposes described herein. Some portion of the embodiments of the present methods include compound analysis techniques applied to gases sampled from target tissue treatment sites.

The tissue compound analysis portion of the embodiments of the present methods may result from gaseous sampling collected from treatment sites not yet subjected to electrosurgical treatment or subjected to treatment from non-electrosurgical systems. Example non-electrosurgical systems include tissue treatment with motorized or mechanical tissue treatment systems, or conventional treatment with scalpel, scissors, or other instruments to cut target tissue such as necrotic and/or infected tissue. Additional examples include treatment of target tissue such as wound tissue with mechanical debridement using the removal of dressing adhered to target tissue such as wound tissue or chemical treatment of target tissue using certain enzymes and other compounds to dissolve target tissue.

The tissue compound analysis portion of the embodiments of the present methods may also result from gaseous sampling collected from treatment sites in situ during electrosurgical treatment or from treatment sites after electrosurgical treatment to determine the state of the target tissue and the progress of treatment. In several embodiments described herein, the state of the target tissue or wound tissue may indicate several characteristics about the target tissue. Analysis of gaseous samples collected from the target tissue may include identification and distinction of the physiological type of tissue present in some embodiments. This analysis is useful in determination of what layer or tissue is being removed or treated or whether tumor tissue or healthy tissue is being treated. Indication of target tissue state in other embodiments relate to the healthy or diseased state of the tissue based on contrast with analysis of known healthy samples or correlative comparison with known diseased state tissue analysis. In other embodiments, determination of target tissue state may indicate the presence of specific types and concentration levels of pathogens or the presence or non-presence of biofilms. In yet other embodiments, determination of the target tissue state may indicate whether a target tissue has been subject to electrosurgical treatment or not.

The assignee of the present invention developed Coblation® electrosurgical technology. Coblation® is an electrosurgical treatment technology that shall serve as an example embodiment electrosurgical treatment for many of the invention embodiments discussed herein. It is understood that other electrosurgical treatment systems and methods as defined above may be employed as well. Similarly, in certain embodiments, the non-electrosurgical treatment systems and methods may also apply to the invention embodiments described herein.

Coblation® involves the application of a high frequency voltage difference between one or more active electrode(s) and one or more return electrode(s) to develop high electric field intensities in the vicinity of the target tissue. The high electric field intensities may be generated by applying a high frequency voltage that is sufficient to vaporize an electrically conductive fluid and form a vapor layer over at least a portion of the active electrode(s) in the region between the tip of the active electrode(s) and the target tissue. The electrically conductive fluid may be a liquid or gas, such as isotonic saline, Ringers' lactate solution, blood, extracellular or intracellular fluid, delivered to, or already present at, the target site, or a viscous fluid, such as a gel, applied to the target site.

When the conductive fluid is heated enough such that atoms vaporize off the surface faster than they recondense, a gas is formed. When the gas is sufficiently heated such that the atoms collide with each other causing a release of electrons in the process, or, the electric field is intense enough to promote the release of electrons from nearby surfaces, an ionized gas or plasma is formed (the so-called “fourth state of matter”). Generally speaking, plasmas may be formed by heating a gas and ionizing the gas by driving an electric current through it, or by shining radio waves into the gas. These methods of plasma formation give energy to free electrons in the plasma directly, and then electron-atom collisions liberate more electrons, and the process cascades until the desired degree of ionization is achieved. A more complete description of plasma can be found in Plasma Physics, by R. J. Goldston and P. H. Rutherford of the Plasma Physics Laboratory of Princeton University (1995), the complete disclosure of which is incorporated herein by reference. Plasma based electrosurgical treatment systems, methods and technology are illustrated and described in commonly owned U.S. Pat. Nos. 6,296,638, 6,589,237; 6,602,248 and 6,805,130 and U.S. patent applications such as U.S. Patent Publication No. 2009/0209958, the disclosures of which are herein incorporated by reference.

In one exemplary embodiment illustrated in FIG. 2, the electrosurgical treatment and compound analysis system (8) includes an electrosurgical treatment probe (10) and a gaseous sampling apparatus (40), and a compound analyzer (60). The electrosurgical treatment probe (10) comprises an elongated shaft (12) and a connector (14) at its proximal end, and one or more active electrodes (16a) disposed on the distal end of the shaft. Also disposed on the shaft but spaced from the active electrode is a return electrode (16b). A handle (20) with connecting power cable (18) and cable connector (22) can be removably connected to the power supply (26).

In the presently described embodiment, an active electrode is an electrode that is adapted to generate a higher charge density relative to a return electrode, and hence operable to generate a plasma in the vicinity of the active electrode when a high-frequency voltage potential is applied across the electrodes, as described herein. Typically, a higher charge density is obtained by making the active electrode surface area smaller relative to the surface area of the return electrode.

Power supply (26) comprises selection means (28) to change the applied voltage level. The power supply (26) can also include a foot pedal (32) positioned close to the user for energizing the electrodes (16a, 16b). The foot pedal (32) may also include a second pedal (not shown) for remotely adjusting the voltage level applied to electrodes (16a, 16b). Also included in the system is an electrically conductive fluid supply (36) with tubing (34) for supplying the probe (10) and the electrodes with electrically conductive fluid. Details of a power supply that may be used with the electrosurgical probe of the currently embodiment is described in commonly owned U.S. Pat. No. 5,697,909, which is hereby incorporated by reference herein.

As illustrated in FIG. 2, the return electrode (16b) is connected to power supply (26) via cable connectors (18), to a point slightly proximal of active electrode (16a). Typically, return electrode (16b) is spaced at about 0.5 mm to 10 mm, and more preferably about 1 mm to 10 mm from active electrode (16a). Shaft (12) is disposed within an electrically insulative jacket, which is typically formed as one or more electrically insulative sheaths or coatings, such as polyester, polytetrafluoroethylene, polyimide, and the like. The provision of the electrically insulative jacket over shaft (12) prevents direct electrical contact between shaft (12) and any adjacent body structure or the surgeon. Such direct electrical contact between a body structure and an exposed return electrode (16b) could result in unwanted heating of the structure at the point of contact causing necrosis.

As will be appreciated, the above-described electrosurgical system and apparatus can applied to wound tissue treatment and equally well applied to a wide range of electrosurgical procedures including open procedures, intravascular procedures, urological, laparoscopic, arthroscopic, thoracoscopic or other cardiac procedures, as well as dermatological, orthopedic, gynecological, otorhinolaryngological, spinal, and neurologic procedures, oncology and the like. Several types of physiological tissue, as defined above, may be treated, both healthy and diseased. However, for several presently-described system embodiments and method embodiments, the electrosurgical treatments are discussed as relating to treat various forms of breaks in skin tissue and chronic surface tissue wounds, including but not limited to skin ulcers, mucus membrane ulcers, foot ulcers including diabetic foot ulcers, cellulitic tissue, venous ulcers, pressure ulcers, surgical wounds, trauma wounds, burns, amputation wounds, wounds exacerbated by immune compromised disease, and wounds associated with radiation and chemotherapy treatments.

The electrosurgical treatment system probe of the presently-described embodiment generates a gas or liquid based plasma in the vicinity of a treatment site. As the density of the plasma or vapor layer becomes sufficiently low (i.e., less than approximately 10²⁰ atoms/cm³ for aqueous solutions), the electron mean free path increases to enable subsequently injected electrons to cause impact ionization within the vapor layer. Once the ionic particles in the plasma layer have sufficient energy, they accelerate towards the target tissue. This ionization, under these conditions, induces the discharge of plasma comprised of energetic electrons and photons from the vapor layer to the surface of the target tissue. Energy evolved by the energetic electrons (e.g., 3.5 eV to 5 eV average energy, with higher-energy electrons in the “tail” of the energy distribution function) can subsequently collide with a molecule and break its bonds, dissociating a molecule into free radicals, which then combine into final gaseous or liquid species. Often, the electrons are accelerated by the electric fields or absorb the radio wave energy by inverse Bremmstrahlung processes, and, because of their small mass do not equilibrate with the heavier ions and, therefore, are hotter than the ions. Thus, the electrons, which are carried away from the tissue towards the return electrode, carry most of the plasma's heat with them, allowing the ions to break apart the tissue molecules in a substantially non-thermal manner. Thus, the target tissue is fragmented. Among the byproducts of this type of ablation are volatile organic compounds (VOCs) and other gases released by the target tissue fragmentation. VOCs emitted from target tissue indicate presence of pathogens, levels of pathogens, presence of biofilms, and indicate types of physiological tissue as discussed below.

By means of this molecular dissociation (rather than thermal evaporation or carbonization), the target tissue structure is volumetrically removed through molecular disintegration of larger organic molecules into smaller molecules and/or atoms, such as hydrogen, oxygen, oxides of carbon, hydrocarbons and nitrogen compounds. This molecular disintegration completely removes the tissue structure, as opposed to dehydrating the tissue material by the removal of liquid within the cells of the tissue and extracellular fluids, as is typically the case with electrosurgical desiccation and vaporization. Further, because the vapor layer or vaporized region has relatively high electrical impedance, it minimizes current flow into the electrically conductive fluid. A more detailed description of these phenomena, termed Coblation®, can be found in commonly assigned U.S. Pat. Nos. 5,683,366 and 5,697,882, the complete disclosures of which are incorporated herein by reference.

In certain embodiments of the present method, the applied high frequency voltage can be used to fragment tissue in several ways, e.g., current can be passed directly into the target site by direct contact with the electrodes such to heat the target site; or current can be passed indirectly into the target site through an electrically conductive fluid located between the electrode and the target site also to heat the target site; or current can be passed into an electrically conductive fluid disposed between the electrodes to generate plasma for treating the target site. In accordance with the present method, the system of FIG. 2 is adaptable to apply a high frequency (RF) voltage/current to the active electrode(s) in the presence of electrically conductive fluid to modify the structure of tissue via liquid based plasma on and in the vicinity of target tissue such as a wound. Thus, with the present method, the system of FIG. 2 can be used to modify tissue by: (1) creating perforations in the chronic wound tissue and in the vicinity of the chronic wound tissue; (2) volumetrically removing tissue (i.e., ablate or effect molecular dissociation of the tissue structure) in the chronic wound tissue and in the vicinity of the chronic wound; (3) forming holes, channels, divots, or other spaces in the chronic wound tissue and in the vicinity of the chronic wound tissue; (4) cutting, resecting, or debriding tissues of the chronic wound and in the vicinity of the chronic wound tissue; (5) inducing blood flow to the tissues of the chronic wound and in the vicinity of the chronic wound tissue; (6) shrinking or contracting collagen-containing connective tissue in and around the chronic wound and/or (7) coagulate severed blood vessels in and around the chronic wound tissue.

In various embodiments of the present method, the electrically conductive fluid possesses an electrical conductivity value above a minimum threshold level, in order to provide a suitable conductive path between the return electrode and the active electrode(s). The electrical conductivity of the fluid (in units of milliSiemens per centimeter or mS/cm) is usually be greater than about 0.2 mS/cm, typically greater than about 2 mS/cm and more typically greater than about 10 mS/cm. In an exemplary embodiment, the electrically conductive fluid is isotonic saline, which has a conductivity of about 17 mS/cm.

It is understood that tissue fragmentation may be accomplished in other embodiments via any electrosurgical treatment or non-electrosurgical treatment in substitution or addition to the liquid plasma embodiment described above. Any electrosurgical treatment or non-electrosurgical treatment may be used prior to the gaseous sampling and analyzer phases of the system illustrated in FIG. 2.

In various embodiments of electrosurgical treatment methods described herein, including the exemplary Coblation® method, the electrosurgical treatment and compound analysis system (8) removes ablation by-products and/or any excess electrically conductive fluid from the surgical treatment site such as a wound bed. In an example embodiment, removal of electrosurgical by-products may be via aspiration. Alternatively, for other electrosurgical treatment or non-electrosurgical treatment techniques, a gas such as VOC may be sampled for exposure to an analyzer. As depicted in the embodiment of FIG. 2, the gaseous sampling instrument (40), may comprise an independent aspiration lumen (42) in fluid communication with other portions of the gaseous sampling apparatus. Alternatively, an integrated aspiration lumen (44) may be integrated into the electrosurgical treatment probe (10) for aspiration of gaseous electrosurgical treatment by-product. Some or all of the samples taken by the gaseous sampling instrument (40) may be gas or vapor in the form of ablation by-product bubbles in fluid, gas produced by the electrosurgical treatment, or gases emitted from the target tissue at the treatment site whether or not active treatment is being administered.

An example type of gas that may be sampled either during electrosurgical treatment, or from emissions from a target tissue site before, after or during electrosurgical treatment includes the volatile organic compounds (VOCs) referenced above. The VOCs sampled by the gaseous sampling instrument (40) may contain a signature combination of molecules that help identify the state of tissue in the target tissue site such as a wound bed. As described in additional detail below, analysis can provide a level of correlation to gas samples from tissue with known tissue status to provide diagnostic identification with varying degrees of certainty. The presence of certain VOC combinations (or other gases) may result from and indicate the electrosurgical treatment itself, for example Coblation® treatment in the present embodiment. Other VOCs or combinations of VOCs indicate physiological tissue type such as the categories of physiological tissue types described above. VOCs emitted from target tissues or generated during electrosurgical or other treatment may also indicate state of the tissue. The state of the tissue may include the presence of infection, biofilm, damaged tissue (e.g., necrotic), disease states, and tumor tissue based on VOCs or combinations of VOCs present. The level of VOCs present may also indicate levels of infection in a particular target tissue. Thus, sampling and analysis of VOCs can provide important diagnostic information before, during, and after the time of treatment of a target tissue to assist in the treatment administered.

Aspiration lumens (42) and (44) may also aspirate small pieces of tissue that are not completely disintegrated by the high frequency energy, or other fluids at the target site, such as blood, mucus, and other body fluids. Accordingly, the various embodiments of the present system include one or more aspiration lumen(s) (42) and (44) in the shaft, or on another instrument, coupled to a suitable vacuum source (not shown) for aspirating fluids from the target treatment site. In various embodiments, the gaseous sampling instrument (40) may also include one or more aspiration active electrode(s) (not shown) coupled to the aspiration lumen for inhibiting clogging during aspiration of tissue fragments from the surgical site.

The gaseous sampling instrument (40) provides separation of the solid pieces of tissue and liquid fluids from the gases to be sampled with a solid and liquid by-product trap (46). The aspiration vacuum draws the ablation by-product through the aspiration lumens (42) or (44) to the solid/liquid by-product trap (46). Gases to be sampled by the system rise to the headspace of the solid/liquid by-product trap (46) or are released from solution into the headspace by passing an inert gas such as nitrogen through the solid/liquid by-product in the trap (46). The aspirated gases are available for removal separate from the solid or liquid by-product in the trap (46) headspace via sampling aperture (48).

In certain embodiments, only gases may be sampled, such as the aspiration of those gases emitted from target tissue sites such as a wound bed. Gases may also be all that is sampled from other tissue fragmentation systems; whether electrosurgical treatments or non-electrosurgical treatments. Exposure to analyzer sensors, such as various “electronic nose” systems described below, may not necessarily require aspiration. In these cases, since no solid or liquid by-product is aspirated, a solid/liquid by-product trap (46) may not be required. Instead, the sampling aperture (48) may be in fluid communication with the headspace over the target tissue via an aspiration lumen (42) or (44). Alternatively, an analyzer sensor may be in fluid communication directly with the headspace over the target tissue with a sampling aperture (48) comprising a sampling interface structure with the analyzer sensor.

Gas sampled from the solid/liquid trap may also be passed through a hydrocarbon moisture trap (50) to remove moisture and prevent contamination of the next stages of the compound analyzer (60). Another description of related aspiration system embodiments can be found in commonly owned U.S. Pat. No. 6,190,381, the complete disclosure of which is incorporated herein by reference for all purposes.

The compound analyzer (60) of the disclosed electrosurgical treatment and compound analysis system (8) receives the gas sampled from the gaseous sampling instrument (40) via a connector and tubing. In an embodiment, the sample gas includes VOCs as described above. The compound analysis system may include one or more compound analysis phases to generate a compound analysis profile. In the example embodiment shown in FIG. 2, the compound analyzer (60) includes gas chromatography phase (GC) (62) and mass spectroscopy phase (MS) (70). Varying types of MS detectors (70) may be employed and include ion mobility spectrometer (IMS), time-of-flight MS (TOF), and quadrupole mass spectroscopy (QMS). In an alternative embodiment of compound analyzer (60), optical methods may be used to detect VOCs. Optical detection systems may employ methods such as ultraviolet (UV) absorption, visible light (VIS) absorption, infrared (IR) absorption by VOCs or other sampled gases to determine a compound analysis profile. In yet another alternative embodiment of compound analyzer (60), fluorescence methods may be used to detect VOCs and prepare a compound analysis profile for the sampled gas. Fluorescent detection systems may employ methods involving application of fluorescing dyes to the target tissue site or wound bed. Then detection of UV excitation, VIS excitation, or IR excitation fluorescence may identify presence and intensity of VOCs in the sampled gases. In an alternate embodiment, optical or fluorescence systems and methods may detect VOCs in vapor-phase molecules sampled from the target tissue bed to generate a compound analysis and determine status of target tissue.

Another alternative embodiment of the compound analyzer (60) includes “electronic nose” systems. Electronic noses are sensitive instruments that detect VOCs and may be used as an alternative compound analyzer (60) to the GC-MS system described in the present embodiment. These instruments are designed to test and discriminate among VOCs without having to identify the individual chemical species present in the volatile mixture. They have an added benefit in that they are portable and have software to sort out the various signatures of sniffed VOCs to provide a compound analysis profile.

There are a range of “electronic nose” sensor technologies including conducting-polymer sensors, metal oxide sensors, metal-oxide silicon field-effect sensors, piezoelectric crystals, optical sensors, and electrochemical sensors. Use of these “electronic noses” have some common operational steps where an electronic sensor array picks up a signal from the sampled VOCs, the information is preprocessed, and then pattern recognition software is applied to identify what bacteria are associated with the detected VOCs. This identification may result from a “learning” process whereby VOCs are analyzed from a known compound or a combination of compounds and the resulting analysis is stored in a library. An extensive library will allow identification of a wide range of compounds.

Examples of three types of electronic nose are: AromaScan A32S® from Osmetech Inc., Libranose 2.1® from Technobiochip Inc., and PEN3® from Airsense Analytics. The AromaScan A32S® from Osmetech Inc. is an organic matrix-coated polymer-type 32 sensor array. AromaScan sensor responses are measured as a percentage of electrical resistance changes to current flow in the sensors, relative to a baseline resistance. The type of polymer can be varied to customize the sensor response.

The Libranose 2.1 from Technobiochip Inc. has eight chemical quartz microbalance sensors. These microbalance sensors are ultrasensitive and capable of measuring small changes in a mass on a quartz crystal. The crystals are oscillated with a voltage and the resonant frequency is sensed. VOCs may be identified depending on the mass sensed.

The PEN3 from Airsense Analytics uses ten metal oxide semiconductor sensors. The metal oxide semiconductor sensors are doped semiconductors that sense the oxygen exchange between the VOCs and the metal coating material of the sensor upon proximity of the VOC molecular gas with the sensor.

Returning to the GC-MS compound analyzer (60) illustrated in FIG. 2, the gas chromatographer (GC) (62) of the example compound analyzer embodiment (60) illustrated in FIG. 2 includes an input connector port (64) at the proximate end of the GC (62) for injection of the gas sample into the GC phase (62) of the analyzer. A carrier gas, often an inert gas such as helium (not shown), may be injected along with the gas sample into the GC phase (62) as well to create a consistent flow of the gaseous sample through the GC phase. The GC connector port (64) is in fluid communication with a capillary column (66). Due to the desirability of a long GC column (66) to separate the gaseous molecular components of the gas sample by various molecular properties as the sample travels through the capillary column, the capillary column appears (66) as a coil inside a temperature regulated environment (68) such as a GC oven. The separation of the gaseous molecular components causes phases of components to arrive at the distal end of the column (66) taking different times to travel the length of the column (66). The time taken to travel the length of the capillary column (66) is referred to as the retention time.

The capillary column (66) is connected at the proximate end of the GC phase (62) to the MS phase (70) of the compound analyzer (60) of the example embodiment. In the example embodiment, the separated gas sample is received at the MS phase (70) from the capillary column (66) of the GC phase (62). The MS phase (70) includes an ionizer (72) to capture and ionize the gaseous molecular components of the gas sample as they arrive from the capillary column (66). The ionizer may be an electron-impact ionization source in one embodiment or other ionization methods to ionize the gaseous sample (e.g., VOCs). A focuser (74) accelerates the portions of ionized gas sample into the deflector (76) and detector (78) of the MS phase (70) of the compound analyzer embodiment (60). The deflector (76) includes charged plates to create an electric and/or magnetic field that separate the ionized portions of the gaseous sample as they arrive at the MS phase (70) by mass-to-charge ratios. These separated ionized components are then detected at the detector (78) and data counts (intensity) and retention time are reported to a computer processing system (84) via data port (80) connected. The data port (80) is connected to an input port (82) on the computer processing system (84) via a cable, wireless connection, infrared connection, or other data connection. The computer processing system (84) then processes and prepares the data received from the detector (78).

In the example embodiment, compound analysis profiles (86) of the gaseous sample are developed and displayed as a function of intensity level (e.g., in nanograms) per retention time (e.g. in minutes) by the computer processing system (84). In alternative embodiments, compound analysis profiles derived from “electronic nose” systems may be used although they may not specifically identify each individual compound. Instead, these systems can take a broader analysis of a plurality of signature VOCs to determine a qualitative tissue state. The compound analysis profiles (86) may also include a table describing the detected compounds according to charge-to-mass ratios and retention times commonly measured by the compound analyzer (60) of the present embodiment. A determination of intensity levels for one or more compounds may also be provided in the compound analysis profiles.

Comparison between the compound analysis profile (86) measured in the gas sample and a database of known compound analysis profiles stored in a database (not shown) may be made by the computer processing system (84) as well. Correlation of profile data measured from the gaseous sample and known compound analysis profiles may be made to yield an estimation of the composition of the sampled target tissue from which the VOC or other gas sample was collected. For example, if a measured compound analysis profile (86) from the gas sample taken from a wound bed is found to correlate 85% with the signature compounds of a known compound analysis profile of an MRSA infected wound, then the computer processing system (84) may provide a diagnosis of the wound bed tissue corresponding to an 85% correlation to MRSA infection. A measure of comparative correlation provides a measure of certainty in the diagnosis made with the VOC compound analysis. Intensity levels of peaks or combinations of peaks that are signatures to MRSA may also provide data to relate potential infection levels or concentrations in pathogen colony forming units (CFU).

In an alternative embodiment, the computer processing system may compare the compound analysis profile (86) from a gas sample from a wound bed with that of a gas sample emitted from known healthy tissue of a similar type to the wound (e.g., a contra-lateral foot without a chronic wound). Comparison of the healthy tissue may be used to determine the disease state of the wound bed based on the correlation of gas samples from the wound bed contrasted with those gathered from the known healthy tissue. Further compound analysis processing embodiments by the computer processing system (84) to assist in treatment diagnoses are discussed below.

Examples of one embodiment of an electrosurgical treatment apparatus that can be used to fragment and treat tissue in accordance with the present method are illustrated in FIGS. 3, and 4A-D. FIGS. 3 and 4A-D show example embodiments of a Coblation® wand. In certain embodiments of the present method, a single electrode (FIG. 3) or an electrode array of plural electrodes (FIGS. 4A-D) may be disposed over a distal end of the shaft of the electrosurgical instrument to generate the plasma that is subsequently applied to the target tissue. In most configurations, the circumscribed area of the electrode or electrode array will generally depend on the desired diameter of the perforations and amount of tissue debriding to be performed. In one embodiment, the area of the electrode array is in the range of from about 0.10 mm² to 40 mm², preferably from about 0.5 mm² to 10 mm², and more preferably from about 0.5 mm² to 5.0 mm.

In addition, the shape of the electrode at the distal end of the instrument shaft will also depend on the size of the chronic wound tissue surface area or other target tissue treatment site. For example, the electrode may take the form of a pointed tip, a solid round wire, or a wire having other solid cross-sectional shapes such as squares, rectangles, hexagons, triangles, star-shapes, or the like, to provide a plurality of edges around the distal perimeter of the electrodes. Alternatively, the electrode may be in the form of a hollow metal tube or loop having a cross-sectional shape that is round, square, hexagonal, rectangular, or the like. The envelope or effective diameter of the individual electrode(s) ranges from about 0.05 mm to 6.5 mm, preferably from about 0.1 mm to 2 mm. Furthermore, the electrode may in the form of a screen disposed at the distal end of the shaft and having an opening therethrough for aspiration of excess fluid and ablation byproducts.

With reference to FIG. 3, in one embodiment the apparatus utilized in the present method comprises an active electrode (316a) disposed on the distal end of a shaft (312). Spaced from the active electrode is a return electrode (316b) also disposed on the shaft (312). Both the active and return electrodes are connected to a high frequency voltage supply (not shown). Disposed in contact with the active and return electrodes is an electrically conductive fluid (320). In one embodiment the electrically conductive fluid forms an electrically conductive fluid bridge (322) between the electrodes. Target tissue bed 110 is treated upon application of a high frequency voltage across the electrodes (316a, 316b) wherein plasma is generated as described above. The generated plasma is used for treating target tissue, such as wound tissue, in accordance with the present embodiment method. The healthy target tissue in this example embodiment is layered epithelial tissue with an epidermal layer (112), a dermal layer (114), and a subcutaneous layer (116). By-product gas and fluid (330) from the electrosurgical treatment is collected in integrated aspiration lumen (344) as shown integrated within distal end shaft (312). A more detailed description of the operation of the electrode configuration illustrated in FIG. 3 can be found in commonly assigned U.S. Pat. No. 6,296,638, the complete disclosure of which is incorporated herein by reference. Advantageously, as the tip of the electrode (316a) presents a relatively broad surface area, such that the electrode tip illustrated in FIG. 3 is beneficially used for treating larger wound areas, including debriding large amounts of dead or necrotic tissue, in accordance with various embodiments of the present method. Smaller pointed surface electrode tip electrosurgical treatment tools are also contemplated and disclosure can be found in commonly assigned U.S. Pat. No. 6,602,248, the complete disclosure of which is incorporated herein by reference. Such a smaller pointed tip electrode may be beneficially used for perforating smaller areas of tissue in the vicinity of the wound tissue to induce blood flow to the tissue

With reference to FIG. 4A, in one embodiment an electrosurgical instrument such as apparatus (410) is utilized in the present method and comprises shaft (412) having a shaft distal end portion (412a) and a shaft proximal end portion (412b), the latter affixed to handle (420). An integrated aspiration tube (444), adapted for coupling apparatus (410) to a vacuum source, is joined at handle (420). An electrically insulating electrode support (408) is disposed on shaft distal end portion (412a), and a plurality of active electrodes (416a) are arranged on electrode support (408). An insulating sleeve (418) covers a portion of shaft (412). An exposed portion of shaft (412) located between sleeve distal end and electrode support (408) defines a return electrode (416b).

Referring now to FIG. 4B, a plurality of active electrodes (416a) are arranged substantially parallel to each other on electrode support (408). In an embodiment for treating wound tissue, active electrodes (416a) may usually extend away from electrode support (408) to facilitate debridement, resection and ablation of tissue, and are particularly configured for debriding large amounts of dead or necrotic tissue. A void within electrode support (408) defines aspiration port of the integrated aspiration lumen (444). Typically, the plurality of active electrodes (416a) span or traverse aspiration port (444), wherein aspiration port (444) is substantially centrally located within electrode support (408). Integrated aspiration lumen (444) is in fluid communication with the gaseous sampling apparatus (40) and a compound analyzer (e.g., (60) of FIG. 2) for aspirating by-product and emitted gaseous materials from a treatment site for separation and analysis.

Referring now to FIG. 4C, a cross-sectional view of apparatus (410) is shown. Aspiration lumen (444) is in fluid communication with its proximal end (444a) and gaseous sampling apparatus (40) (see FIG. 2). Aspiration port, channel, and tube (444) provide a suction unit or element for drawing gases to be analyzed as well as fluid and pieces of tissue toward active electrodes (416a) for further ablation after they have been removed from the target site. Aspiration tube (444) removes unwanted materials such as ablation by-product gases, blood, or excess saline from the treatment site. Handle (420) houses a connection block (405) adapted for independently coupling active electrodes (416a) and return electrode (416b) to a high frequency power supply. An active electrode lead (421) couples each active electrode (416a) to connection block (405). Return electrode (416b) is independently coupled to connection block (405) via a return electrode connector (not shown). Connection block (405) thus provides a convenient mechanism for independently coupling active electrodes (410) and return electrode (416b) to a power supply (e.g., power supply 26 in FIG. 2). In alternative embodiments, the active electrodes may be arranged in a screen electrode configuration, as illustrated and described in commonly owned U.S. Pat. Nos. 6,254,600 and 7,241,293, the disclosures of which are herein incorporated by reference.

Referring now to FIG. 4D, apparatus (410) is characterized by outer sheath (452) external to shaft (412) to provide an annular fluid delivery lumen (450) in certain embodiments. The distal terminus of outer sheath (452) defines an annular fluid delivery port (456) at a location proximal to return electrode (416b). Outer sheath (452) is in fluid communication at its proximal end with fluid delivery tube (454) at handle (420). Fluid delivery port (456), fluid delivery lumen (450), and tube (454) provide a fluid delivery unit for providing an electrically conductive fluid (e.g., isotonic saline) to the distal end of apparatus (410) or to a target site undergoing treatment. To complete a current path from active electrodes (416a) to return electrode (416b), electrically conductive fluid is supplied therebetween, and may be continually resupplied to maintain the conduction path. Provision of electrically conductive fluid may be particularly valuable in a dry field situation (i.e., in situations where there are insufficient native electrically conductive bodily fluids). Alternatively, delivery of electrically conductive fluid may be through a central internal fluid delivery lumen, as illustrated and described in commonly owned U.S. Pat. Nos. 5,697,281 and 5,697,536, the disclosures of which are herein incorporated by reference.

In a typical procedure involving treatment of a chronic wound according to an embodiment of the present method, it may be necessary to use a series of electrosurgical treatments in combination with compound analysis to determine progress and next steps for treatment of the wound. For example, in a first step, an electrode of the type illustrated in either FIG. 3 or 4A-D may be employed to debride unhealthy or necrotic tissue comprising and surrounding the chronic wound site and wound bed. In a second step of the treatment, analysis of gaseous by-product from the debridement, or alternatively, analysis of gases emitted from wound bed locations after debridement provide diagnostic feedback relating to the electrosurgical debridement procedure. Comparison may be made between gaseous by-product analysis in situ during treatment and post-treatment emitted gases to determine progress of the debridement procedure. In addition, pre-treatment samples and analysis may be compared with post-treatment samples and analysis, or compared with sampling at any time point during the debridement to check the status of the debridement treatment. Depending on the results of the analysis, further debridement treatment using the same active electrode type or a different electrode configuration may be used to focus the electrosurgical treatment. It is contemplated that the first and second steps described above may be performed in any order or sequence such that pre-treatment emitted gases may be analyzed before debridement and/or after debridement as well as analysis of in situ generated gaseous by-products. In another embodiment, progress analysis of the electrosurgical debridement of necrotic tissue and sterilization of the treatment site by removing debris, biofilm, bacteria, and other pathogens with exposure to plasma, both on the periphery of a wound bed and within the wound bed itself, may prepare a bleeding wound bed post-surgical treatment such as wound closure or skin graft application or other treatment. Analysis of gases such as VOCs emitted at wound locations provides valuable diagnostic feedback to assist in determination of the next steps of treatment.

Typically, during debridement procedures that utilize an electrode configuration of the type illustrated in FIGS. 4A-D, apparatus (410) is advanced toward the target tissue such that electrode support (408) is positioned to be in close proximity to the target tissue, while active electrodes (416a) are positioned so as to contact, or to be in closer proximity to, the target tissue. Active electrodes (416a) are particularly effective for debriding tissue because they provide a greater current concentration to the tissue at the target site. The greater current concentration may be used to aggressively create a plasma within the electrically conductive fluid, and hence a more efficient debridement of tissue at the target site. In use, active electrodes (416a) are typically employed to ablate tissue using the Coblation® mechanisms as described above. Voltage is applied between active electrodes (416a) and return electrode (416b) to volumetrically loosen fragments from the target site through molecular dissociation. Once the tissue fragments are loosened from the target site and gases (e.g., VOCs) are released, the tissue fragments can be ablated in situ with the plasma (i.e., break down the tissue by processes including molecular dissociation or disintegration), removed along with gases and fluids via an aspiration lumen, or removed via irrigation or other suitable method. As a result, electrosurgical apparatus (410) preferably removes unhealthy or necrotic tissue and debris, biofilm, bacteria, and other pathogens, both on the periphery of the wound and within the wound bed itself. This is done in a highly controlled manner when treatment progress and wound tissue status may be analyzed and diagnosed during treatment or shortly before or after electrosurgical treatment. This produces a more uniform, smooth, and contoured tissue surface with indication that the surface is at an improved health status that promotes sterilization and is more conducive to proper healing. Alternatively and in addition, in certain embodiments it may be desirable that small severed blood vessels at or around the target site are typically simultaneously coagulated, cauterized and/or sealed as the tissue is removed to continuously maintain and invoke hemostasis during the procedure.

Applicants believe that the presently-described methods of treatment, VOC sample collection, and compound analysis for wound tissue utilizing the above-referenced electrosurgical devices, gas sample collection devices, and analyzer devices evokes a more organized and coordinated healing response than is typically associated with wound treatments. Specifically, the application of high frequency voltage and resulting plasma to wound tissue for debridement, in conjunction with analysis of pre-treatment, in situ, or post-treatment tissue status using compound analysis techniques to gases such as VOCs retrieved from the treatment site provides critical information relating to progress of electrosurgical treatment. This permits diagnosis for more accurate next steps of treatment of the wound.

The voltage difference applied between the return electrode(s) and the return electrode is high or radio frequency, typically between about 5 kHz and 20 MHz, usually being between about 30 kHz and 2.5 MHz, preferably being between about 50 kHz and 500 kHz, more preferably less than 350 kHz, and most preferably between about 100 kHz and 200 kHz. The RMS (root mean square) voltage applied will usually be in the range from about 5 volts to 1000 volts, preferably being in the range from about 10 volts to 500 volts depending on the active electrode size, the operating frequency and the operation mode of the particular procedure or desired effect on the tissue (e.g., contraction, coagulation, cutting or ablation).

Typically, the peak-to-peak voltage for ablation or cutting of tissue will be in the range of from about 10 volts to 2000 volts, usually in the range of 200 volts to 1800 volts, and more typically in the range of about 300 volts to 1500 volts, often in the range of about 500 volts to 900 volts peak to peak (again, depending on the electrode size, the operating frequency and the operation mode). Lower peak-to-peak voltages will be used for tissue coagulation or collagen contraction and will typically be in the range from 50 to 1500, preferably from about 100 to 1000, and more preferably from about 120 to 600 volts peak-to-peak.

FIGS. 5A-5D depict example compound analysis profiles from a GC-MS analyzer (e.g., (60) in FIG. 2). Each compound analysis profile is shown with retention time in minutes on the x-axis (510) and intensity (or amplitude) in nanograms (ng) on the y-axis (520). Peaks (530) are depicted with the retention time and correspond to molecular gaseous components of the gas sample based on mass-to-charge ratios. Each compound analysis profile relates to experimental data for various cases. One of skill in the art would understand similar data for human tissue is acquired through similar methods.

FIG. 5A depicts a compound analysis profile of a combination Escherichia coli (E. coli) and Psuedomonas aeruginosa (Pseudomonas) bacterial infection embedded in biofilm on a preparation of pigskin. Sample VOC gas emitted from the infected biofilm/pigskin sample is gathered prior to treatment by aspiration lumens (42) or (44) of the gaseous sampling apparatus (40). These VOC samples were analyzed by the compound analyzer (60). FIG. 5B depicts a compound analysis profile of the same combination E. coli and Psuedomonas aeruginosa bacterial infection embedded in biofilm on pigskin after treatment with a Coblation® electrosurgical system. The following Table 1 shows preparations for gaseous analysis using a device similar to the system 8 depicted in FIG. 2. FIG. 5A depicts Sample 6 and FIG. 5B depicts Sample 4 in Table 1. FIG. 5C depicts Sample 9 and FIG. 5D depicts Sample 2 in Table 1. The bacterial preparations selected include several bacterial pathogens commonly found in wound tissue beds.

TABLE 1

| | | | | Incubation | |
|---|---|---|---|---|---|
| Sample | Medium | Inoculation | CFUs | period | |
| 1 | Blood agar | Streptococcus | 2000 | 4 | days |
| | BIOFILM | pyogenes | | | |
| 2 | Blood agar | MRSA + Strep. | 1000 + 1000 | 24 | hr. |
| 3 | Pigskin (in-vitro) | Control | Control | 12 | hrs. |
| 4 | Pigskin (in-vitro) | E. coli + | 1000 | 4 | days |
| | BIOFILM following | pseudomonas | | | |
| | Coblation | | | | |
| 5 | Blood agar | E. coli + | 1000 + 1000 | 4 | days |
| | BIOFILM | pseudomonas | | | |
| 6 | Pigskin (in-vitro) | E. coli + | 1000 + 1000 | 4 | days |
| | BIOFILM | pseudomonas | | | |
| 7 | Pigskin (in-vitro) | Streptococcus | 1000 | 12 | hrs. |
| | | pyogenes | | | |
| 8 | Pigskin (in-vitro) | Streptococcus | 1000 | 12 | hrs. |
| | following | pyogenes | | | |
| | Coblation | | | | |
| 9 | Silicone pad | MRSA | 1000 | 4 | days |
| | BIOFILM | | | | |
| | COBLATION | | | | |
| | HEADSPACE | | | | |

In Tables 2A and 2B (below), corresponding data relating to the compound analysis profiles, including retention time peaks (530), shown in FIGS. 5A-D as well as other compound analysis profiles. Tables 2A and 2B provide description of corresponding gaseous components detected. Components resulting from Coblation® or present post-Coblation are denoted with an asterisk.

TABLE 2A

| | RT | Sample 1 | Sample 2 | Sample 3 | Sample 4 | Sample 5 | Sample 6 |
|---|---|---|---|---|---|---|---|
| | (min) | (ng) | (ng) | (ng) | (ng) | (ng) | (ng) |
| sulfur dioxide | 1.9 | — | 0.87 | — | 0.55 | — | — |
| Ethanol | 2.45 | 18.7 | 8.98 | 16.6 | 21.2 | 19.5 | 22.7 |
| Acetonitrile* | 2.59 | — | — | — | 2.02 | — | — |
| Acetone | 2.7 | 42 | 7.24 | 176 | 3.48 | 9.45 | 11.71 |
| 2-propanol | 2.81 | 101 | 15.1 | 636 | 52.6 | 7.66 | 72.8 |
| 2-propenenitrile* | 2.97 | — | — | — | 0.97 | — | — |
| 2-methyl-1,3-butadiene | 3.06 | — | 0.34 | — | — | — | — |
| Thiobismethane | 3.14 | — | 0.24 | — | — | 2.54 | — |
| Dichloromethane | 3.24 | — | 0.9 | — | — | — | — |
| methylsulfonylmethane | 3.51 | 0.81 | — | — | — | — | — |
| 2-methylpropanal* | 3.62 | — | — | — | — | — | — |
| 3-ammino-1-propine* | 3.7 | — | — | — | — | — | — |
| 2-methyl-2-propenal* | 3.83 | — | — | — | — | — | — |
| vinyl acetate | 4.11 | — | — | 8.33 | — | — | — |
| 2-butanone | 4.33 | 1.35 | 1.36 | — | — | 1 | — |
| 2-butanol | 4.62 | — | — | — | — | — | — |
| 2-methyl-propanenitrile* | 4.78 | — | — | — | — | — | — |
| acetic acid ethyl ester* | 4.9 | — | — | — | — | — | — |
| Tirchloromethane | 4.94 | 0.65 | 1.54 | 0.48 | — | — | — |
| 2-methyl-1-propanol | 5.29 | — | 1.53 | — | — | — | — |
| 3-methylbutanal | 5.82 | — | 1.31 | 0.73 | — | — | — |
| 2-methylbutanal* | 6.11 | — | — | — | — | — | — |
| Benzene | 6.23 | — | 0.48 | 0.75 | 0.52 | 0.35 | — |
| iso-butanonitrile* | 7.52 | — | — | — | — | — | — |
| 3-methyl-1-butanol | 8.1 | — | 79 | — | — | — | — |
| 4-methyl-2-pentanone | 8.15 | 4.83 | — | 2.72 | 0.81 | 4.61 | 0.54 |
| 2-methylbutan-1-ol | 8.22 | — | 5.34 | — | — | — | |
| Dimethyldisulfide | 8.32 | 0.32 | 4.93 | — | — | 13 | 1.03 |
| Toluene | 9.08 | 0.38 | — | — | 0.41 | 0.43 | — |
| toluene + | 9.08 | — | — | 0.55 | — | — | — |
| unknown[43, 100, 281, 45] | | | | | | | |
| hexamethylcyclotrisiloxane | 10.67 | 7.35 | 3.11 | 0.42 | 3.55 | 5.12 | 3.73 |
| 4-methyloctane | 10.76 | 0.9 | 0.45 | — | — | 0.76 | |
| 1-propoxy-2-propanol | 10.9 | — | — | — | 0.6 | — | 0.65 |
| Octane | 11.72 | 0.32 | — | — | — | 0.32 | — |
| Cyclohexanone* | 11.83 | — | — | — | — | — | — |
| Benzaldehyde | 13.37 | 0.77 | 1.14 | 1.59 | 0.85 | 0.87 | 0.32 |
| Phenol | 13.69 | — | — | 0.58 | — | — | — |
| Dimethyltrisulfide | 13.74 | — | 1.02 | — | — | — | — |
| octamethylcyclotetrasiloxane | 14.45 | 1.53 | 1.03 | 0.43 | 0.43 | 0.95 | 0.51 |
| 2-ethyl-1-hexanol | 14.86 | — | 29.1 | — | — | — | — |
| 4-methyldecane | 15.06 | — | 2.04 | 0.42 | — | — | — |
| 1-phenylethanone | 15.52 | — | — | 0.92 | — | — | — |
| 2,4,6-trimethyldecane | 15.78 | 1 | 1.44 | 0.86 | 0.62 | 1.07 | 0.66 |
| 1-undecene | 16.29 | — | — | — | — | 0.74 | — |
| decamethylcyclopentasiloxane | 17.43 | 1.78 | 2.57 | 1.36 | 0.75 | 1.54 | 0.7 |
| Alkane | 19.64 | 0.84 | 0.72 | 0.6 | 0.55 | 1.2 | 0.49 |

TABLE 2B

| | RT | Sample 7 | Sample 8 | Sample 9 |
|---|---|---|---|---|
| Compound | (min) | (ng) | (ng) | (ng) |
| sulfur dioxide | 1.9 | — | — | — |
| Ethanol | 2.45 | 18.9 | 42.6 | 23.4 |
| Acetonitrile* | 2.59 | — | 7.23 | 5.6 |
| Acetone | 2.7 | 35 | 174 | 6.31 |
| 2-propanol | 2.81 | 182 | 630 | 55.4 |
| 2-propenenitrile* | 2.97 | — | — | 2.22 |
| 2-methyl-1,3-butadiene | 3.06 | — | — | — |
| Thiobismethane | 3.14 | — | — | — |
| Dichloromethane | 3.24 | — | — | — |
| methylsulfonylmethane | 3.51 | — | 2.21 | — |
| 2-methylpropanal* | 3.62 | — | 1.28 | 3.47 |
| 3-ammino-1-propine* | 3.7 | — | 1.77 | 1.24 |
| 2-methyl-2-propenal* | 3.83 | — | — | 0.81 |
| vinyl acetate | 4.11 | 2.4 | — | — |
| 2-butanone | 4.33 | — | — | — |
| 2-butanol | 4.62 | — | — | 0.86 |
| 2-methyl-propanenitrile* | 4.78 | — | — | 0.62 |
| acetic acid ethyl ester* | 4.9 | — | 0.59 | — |
| Tirchloromethane | 4.94 | 0.61 | 0.38 | — |
| 2-methyl-1-propanol | 5.29 | — | — | — |
| 3-methylbutanal | 5.82 | — | 1.47 | 4.49 |
| 2-methylbutanal* | 6.11 | — | 0.49 | 1.57 |
| Benzene | 6.23 | 0.6 | 0.84 | 0.92 |
| iso-butanonitrile* | 7.52 | — | — | 0.74 |
| 3-methyl-1-butanol | 8.1 | — | — | — |
| 4-methyl-2-pentanone | 8.15 | 1.59 | 2.09 | 1.22 |
| 2-methylbutan-1-ol | 8.22 | — | — | — |
| Dimethyldisulfide | 8.32 | — | 0.27 | — |
| Toluene | 9.08 | 0.34 | 1.16 | 0.74 |
| toluene + unknown[43, 100, | 9.08 | — | — | — |
| 281, 45] | | | | |
| hexamethylcyclotrisiloxane | 10.67 | 2.18 | 6.55 | 0.8 |
| 4-methyloctane | 10.76 | — | — | — |
| 1-propoxy-2-propanol | 10.9 | — | — | — |
| Octane | 11.72 | — | — | — |
| Cyclohexanone* | 11.83 | — | 1.51 | 1.12 |
| Benzaldehyde | 13.37 | 0.99 | 1.53 | 0.8 |
| Phenol | 13.69 | — | 0.73 | — |
| Dimethyltrisulfide | 13.74 | — | — | — |
| octamethylcyclotetrasiloxane | 14.45 | 0.36 | 1 | — |
| 2-ethyl-1-hexanol | 14.86 | — | — | — |
| 4-methyldecane | 15.06 | — | 0.38 | — |
| 1-phenylethanone | 15.52 | — | 0.77 | — |
| 2,4,6-trimethyldecane | 15.78 | 0.62 | 0.65 | 0.8 |
| 1-undecene | 16.29 | — | — | — |
| decamethylcyclopentasiloxane | 17.43 | 1.81 | 1.89 | 0.98 |
| Alkane | 19.64 | 0.68 | 0.71 | 1.25 |

As can be seen in Table 2A, limited change occurred between the compound analysis profiles of FIGS. 5A (Sample 6) and 5B (Sample 4) pretreatment compared to post-treatment with a Coblation® electrosurgical system. The infection indicator 1-propoxy-2-propanol (intensity of 0.65 in Sample 6 and 0.60 Sample 4) of combination E. coli and Psuedomonas aeruginosa bacterial infection embedded in biofilm on pigskin is reduced, but largely unchanged. This potentially indicates additional electrosurgical treatment is necessary. Coblation® signatures such as acetonitrile and 2-propenenitrile appear in Sample 4 for the compound analysis of FIG. 5B after Coblation® but not before in Sample 6.

FIG. 5C shows a gas sample taken from headspace above a treatment site during Coblation® treatment of a preparation of Staphylococcus aureus (MRSA) on silicone described in Table 1 as Sample 9. Compound analysis profile data for Sample 9 in Table 2B shows the combination of electrosurgical treatment signatures and treated MRSA infection signatures. At peaks (530) of FIG. 5C with retention times of 3.63 and 5.83, the 2-methylproanal and 3-methylbutanal signatures from Coblation® treatment as well as other Coblation® signatures can be seen for Sample 9 (asterisks in Table 2B).

FIG. 5D shows a compound analysis profile for a gas sample taken from emitted gas above a treatment site before any electrosurgical treatment. The treatment site is a preparation on agar infected with a combination of MRSA and Streptococcus pyogenes (Strep) and described in Table 1 as Sample 2. Sample 2 in Table 2A shows the compound analysis profile data of the gaseous sample shown in FIG. 5D. FIG. 5D shows MRSA signature peaks (530) at retention time 8.10 for 3-Methyl-2-Butanol and at retention time 14.86 for 2-Ethyl-1-Hexanol. Table 2A shows the respective intensities (in ng) for these peaks of Sample 2 as well as other detected components in the gaseous sample analyzed. Peaks (530) at retention time 2.82 for isopropyl alcohol or 2-propanol (IPA) of FIG. 5D indicate a combination signature of MRSA and Strep when in connection with other indicators of those bacteria. 2-Methylbutan-1-ol at peak (530) with retention time of 8.22 and dimethyldisulfide (DMDS) at peak (530) with retention time of 8.32 are additional indicators of the MRSA/Strep pathogen combination.

FIGS. 5A-D and data in Tables 2A-B may comprise an example embodiment of known compound analysis profiles in a database for comparison by a computer processing system such as (84) of the compound analyzer (60) shown in embodiment of FIG. 2. While the above profiles depict test examples of pathogens on agar, silicone, and pigskin which may be useful for correlative diagnosis, one of ordinary skill can appreciate that similar profiles may also be stored for human target tissues with or without pathogen infections and biofilms. These human target tissue profiles comprise additional embodiments of the databases of known compound analysis profiles used in diagnostic correlation. Additionally, it is appreciated that known compound analysis profiles may be stored in the database for any stage of treatment including pre-treatment (or no treatment) measurements, in situ treatment measurements, and post-treatment measurements. The known compound analysis profiles from in situ treatment and post-treatment may further include profiles having signatures indicating any of the variety of energy-based electrosurgical treatments as described above, including Coblation® treatment.

With reference to FIG. 6, the present method in one embodiment is a flow chart of a procedure for treating and analyzing wound tissue to facilitate further treatment. In this particular embodiment, the analysis is in situ with treatment. It is appreciated that alternatively, however, analysis of emitted gases (e.g., VOCs) may be conducted by the analysis portions of the method embodiment of FIG. 6 independent of the treatment portions of the recited method embodiment. In particular embodiments, the method (600) starts at (601) and proceeds to block (605) where an energy-based electrosurgical treatment instrument or transmission is positioned in close proximity to the chronic wound tissue. In an example embodiment, an active electrode is positioned proximately to the wound tissue. At (610), the method exposes the wound bed to electrosurgical treatment at a treatment site of the target tissue. At block (615), this electrosurgical treatment exposure fragments tissue from the wound bed and generates gaseous by-products such as VOCs in addition to liquid or solid by-product. In an example embodiment, tissue fragmentation may be done by applying a high-frequency voltage between an active electrode and a return electrode sufficient to develop a high electric field intensity associated with a vapor layer proximate the active electrode. Proceeding to (620), the generated gaseous by-product is separated from the liquid and solid by-product by a liquid/solid by-product trap (46) as shown in the FIG. 2 system embodiment. A sampling aperture, such as (48) in FIG. 2, gathers the separated molecular gaseous sample for analysis by the analyzer at (625). In one embodiment, at least a major component of the molecular gaseous sample are volatile organic compounds (VOCs) drawn in situ from the treatment site.

At (630), the gaseous by-product sample is injected into an analyzer, such as GC-MS analysis or an analyzer utilizing an electronic nose system such as those described above for detecting VOCs. The detector of the analyzer system provides data relating to the components of the gaseous by-product sample to a processing system for determination of a compound analysis profile at (635). The processor may then make a comparison at (640) to correlate the measured compound analysis profile with a database of known compound analysis profiles. Correlative analysis may be done at (645) to provide an estimate of the match with the known profiles. For example, a range of correlation between the compound analysis profile of data table entries (or peaks) of the measured gaseous by-product sample may be made. The correlation range may reflect a determination of how close to a 100% match the compound analysis profile of the measured sample is to the known compound analysis profile signatures. The known compound analysis profiles correspond to tissue status characteristics such as tissue types, pathogens, and biofilms. The percentage correlation provides an indication of certainty of the diagnostic match.

Proceeding to (650), a diagnosis correlation with the known compound analysis profile is provided to assist with determination of future treatment action, if any. It provides an indication of wound tissue status relatively concurrently with the electrosurgical treatment. At (655), intensity levels of signature peaks or table entries may also diagnose infection levels for pathogens present at the treatment site.

Referring now to FIG. 7, another flowchart embodiment for a procedure to treat target tissue and analyze gaseous samples emitted (e.g., VOCs) from a target tissue is illustrated. The gaseous sample analysis permits efficient diagnosis of target tissue state or infection thereby facilitating the treatment of the target tissue. The target tissue may be chronic wound tissue or other tissue to be electrosurgically treated. In particular embodiments, the method (700) starts at (701) and proceeds to block (725) where an aspiration lumen, such as (42) or (44) from FIG. 2, and a sampling aperture, such as (48) in FIG. 2, gather an emitted gaseous sample from a target tissue location. In the first pass of the method depicted in the FIG. 7 flowchart, the treatment site either has not been treated yet or will not be treated. At (730), the emitted gaseous sample gathered from the treatment site is injected into an analyzer, such as a GC-MS analyzer. In an alternative embodiment, the collected gas is exposed to an electronic nose system analyzer such as those described above for detecting VOCs. The analyzer has a detector that provides data relating to the components of the gaseous by-product sample to a processing system. The processing system determines a compound analysis profile of the gaseous sample from the target tissue at (735). The processor may then make a comparison at (740) to correlate the measured compound analysis profile of the gas sample with a database of known compound analysis profiles. The known compound analysis profiles indicate a potential state of the wound tissue, such as an infection type present. The processor may also compare the measured compound analysis profile with other measured compound analysis profiles at (745). This may include comparison to a gas sample taken and analyzed from a known healthy control tissue to determine the relative disease state of the wound. In certain alternative embodiments, the known healthy control tissue may be taken from a location on the patient away from the wound site on the same patient. In one particular embodiment in which the wound of the patient is located on the patient's limb, the known healthy control tissue may be selected to be from a corresponding location on the opposite limb. As will be seen below, other measured compound analysis profiles may be of gas samples collected during in situ treatment of after treatment such as those measured as described below for second and third passes through the flowchart method (700) of FIG. 7.

When correlating pre-treatment compound analysis profiles of gas samples to known compound analysis profiles at (740), correlation may be made based on known signatures of physiological tissue types, pathogen types, or biofilms. In one embodiment, correlative analysis for diagnosis may include a correlative range of the percentage match values with one or more a known compound analysis profiles. For example, a plurality of known compound analysis profiles for a given tissue status characteristic may be used as a known comparison basis rather than only one compound analysis profile. Thus, the measure compound analysis profile may be compared to a range of expected analysis values corresponding to a tissue state.

The correlation level and the corresponding tissue status are then provided at (745). The correlation level between the measured compound analysis profile data table entries (or peaks) and known compound analysis profile data shows how close that the measured profile is to a 100% match. This, in turn, provides a relative level of certainty that the measured VOCs emitted from the target tissue indicate a characteristic tissue type, pathogen, or biofilm at the target tissue. The above correlation and association with tissue status and characteristics is a diagnosis of the target tissue. The diagnosis assists with determination of the outcome of current treatment and the course of future treatment action, if any. In the described embodiment, the correlative diagnosis provides an indication of wound tissue status relatively concurrently with the electrosurgical treatment in situ or shortly before or after treatment. Similar to the method embodiment shown in FIG. 6, intensity levels of signature peaks or table entries for compounds present in the VOC sample may also diagnose infection levels for pathogens present at the treatment site. A first pass of the method of FIG. 7 ends here and an embodiment the present method may end as well. Alternatively, the method may proceed to block (750).

Proceeding to block (750) from block (735) begins a second pass through the method embodiment (700) of FIG. 7. At (750), target tissue is exposed to electrosurgical treatment at the treatment site in accordance with techniques as described above. Gaseous by-product of the electrosurgical treatment is gathered in situ via aspiration lumen and sampled via sampling aperture at (755). Alternatively, other embodiments may include non-electrosurgical treatments whereby samples are gathered in situ. Techniques and systems for gathering and separating electrosurgical treatment by-products or non-electrosurgical by-products may be used similar to those described above. The flowchart then proceeds back to block (730) where the gathered gaseous by-product sample is injected into an analyzer for determining a compound analysis profile at (735). This compound analysis profile of the in situ gaseous by-product sample is compared to known compound analysis profiles at (740). The analysis processes a correlation between the measured profile and known compound analysis profiles. Following this, the diagnostic association to a known tissue state is made as described before. For example, correlation may indicate a diagnostic association of the target tissue as infected, having biofilm present, being damaged, or having been electrosurgically treated. The association may also identify the physiological tissue type. At (745), the measured gaseous by-product compound analysis profile may also be compared to pre-treatment or post-treatment compound analysis profiles to contrast them and determine progress of the electrosurgical treatment. In another embodiment, a comparison may be made with a previous compound analysis profile of in situ gaseous by-product sampled from an earlier round of electrosurgical treatment. Such a comparison permits assessment of the progress of repeated electrosurgical treatments. In yet another embodiment, comparison may be made with a control profile of healthy tissue sample gases at (745) to determine differences and ongoing disease state of the treated tissue, if any. The second pass may end at this point. An embodiment of the method may end here as well. Alternatively, the method may proceed to block (760).

Proceeding to block (760) begins the third pass of the method embodiment (700). At (760), an aspiration lumen and a sampling aperture gathers a post-treatment emitted gaseous sample from a target tissue location after electrosurgical treatment, or alternatively non-electrosurgical treatment. The flow then proceeds back to block (730) where the post-treatment emitted gaseous sample is injected into an analyzer for determining a compound analysis profile at (735). This compound analysis profile of the post-treatment emitted gaseous sample is compared to known compound analysis profiles at (740) for correlation and diagnosis as described above. The post-treatment compound analysis profile may also be compared to a pre-treatment or in situ measurement compound analysis profiles at (745) to contrast the profiles and determine progress of the electrosurgical treatment. In another embodiment, a comparison may be made with a previous post-treatment emitted gaseous sample from an earlier round of electrosurgical treatment to assess ongoing progress of the rounds of electrosurgical treatment. In yet another embodiment, comparison may be made with a control sample of healthy tissue gases at (745) to determine differences and ongoing disease state of the treated tissue, if any. The third pass of the method embodiment 700 may end at this point.

FIG. 8 illustrates an example embodiment of a wound bed (110) segmented into a grid (820) of target tissue zones (830). A surgical navigation system and detector may be used to provide accurate segmentation of the target tissue treatment site or wound tissue bed (110). There are several types of navigation systems available for use with medical systems such as the treatment and analysis system described herein. One type is electromagnetic (for example, Aurora®, Northern Digital Inc., Ontario, Canada) and another is optical (Medtronic StealthStation®). With electromagnetic navigation and detection, a small tracking box is placed near the patient and then small coils are placed on the instrument to be detected. The instrument tip may then be tracked in three dimensions to better than 1 mm position or 1 degree angulation accuracy. Immobilization of the target tissue site permits calibration of the electromagnetic navigation system relative to locations (830) within the patient space. Navigation is conducted using tracking and display software. Thus, target tissue zones or locations (830) within the wound bed (110) or target tissue treatment site may be determined.

An alternative embodiment includes optical navigation and detection systems. Optical navigation systems use a pair of fixed position cameras that interact with an instrument such as an electrosurgical device having three or more LEDs positioned on the instrument. The LEDs are tracked with about the same accuracy as the electromagnetic systems. Tracking and display software monitors the target tissue zones (830) and instrument location relative to patient space for the treatment site or wound bed (110).

Referring now to FIG. 9, a flowchart embodiment for a procedure to treat target tissue, analyze gaseous samples (e.g., VOCs), and map the diagnoses resulting from analysis of the VOCs is illustrated. The method embodiment (900) facilitates treatment of a target tissue such as a wound tissue bed. The method embodiment begins as (901) and proceeds to (905) where the system segments a target wound bed into wound bed location zones. As described above in connection with FIG. 8, various types of treatment site navigation systems and location detectors may be used to segment the target tissue bed. In one embodiment similar to that illustrated in FIG. 8, the segmentation is in a grid. Other segmentation of a wound bed may be advantageous including 3-D mapping, or segmenting the wound bed into overlapping zones.

Proceeding to (910), molecular gaseous samples for each target tissue zone may be gathered and sampled pre-treatment, in situ during treatment, or post-treatment according to several methods and techniques described above. At (915), the molecular gaseous samples associated with each target tissue zone are injected into a compound analyzer to determine compound analysis profiles for each tissue target zone. Alternatively, the molecular gases may be exposed to an electronic nose compound analyzer embodiment. A computer processor system may then compare the compound analysis profiles for each target tissue zone with known compound analysis profiles. Alternatively, comparison may be made with other measured pre-treatment, in-situ, or post-treatment compound analysis profiles from the same or nearby target tissue zones. Proceeding to (925), the system may then map and display diagnostic results and correlations for each target tissue zone in the target tissue wound bed. At (930), the location of an electrosurgical treatment device, energy-based transmission target, or non-electrosurgical treatment device may be detected by the treatment site navigation system. The location of the electrosurgical treatment device, transmission target, or other device is displayed relative to the diagnostic map of the segmented target tissue bed zones. The location of the fragmentation treatment instrument in the wound bed and the current tissue state diagnosis at that and nearby locations will greatly assist treatment decisions. At decision diamond (935), it is determined whether re-mapping is needed for one or more target tissue zones. Remapping may be necessary due to treatment altering tissue at some target tissue zones. If repeat assessment is desired, the flow returns to block (910) to reassess the compound analysis profile for the zone from a current gaseous sample. If repeat assessment is not required, the method embodiment (900) ends.

While preferred embodiments of this disclosure have been shown and described, modifications thereof can be made by one skilled in the art without departing from the scope or teaching herein. The embodiments described herein are exemplary only and are not limiting. Because many varying and different embodiments may be made within the scope of the present inventive concept, including equivalent structures, materials, or methods hereafter thought of, and because many modifications may be made in the embodiments herein detailed in accordance with the descriptive requirements of the law, it is to be understood that the details herein are to be interpreted as illustrative and not in a limiting sense.

What is claimed is:

1. A method comprising:
exposing a wound bed to electrosurgical treatment to generate fragmented wound tissue in situ;
gathering a molecular gaseous by-product sample of the fragmented wound tissue;
analyzing the molecular gaseous by-product sample of the fragmented wound tissue to generate a fragmented wound tissue compound analysis profile;
comparing the fragmented wound tissue compound analysis profile with a database of known compound analysis profiles; and
providing a diagnosis of the wound tissue based on the comparison of compound analysis profiles.

2. The method of claim 1, wherein the diagnosis is provided to assist in determination of a disease state of the wound tissue during the electrosurgical treatment.

3. The method of claim 1, further comprising:
gathering a molecular gaseous sample emitted from a location on the remaining wound bed after electrosurgical treatment of the wound tissue;
analyzing the molecular gaseous sample emitted from a location on a wound bed after electrosurgical treatment to generate a post-treatment compound analysis profile;
comparing a post-treatment compound analysis profile with a database of known compound analysis profiles; and
providing a post-treatment diagnosis of the remaining wound tissue at the wound bed location based on the comparison of the post-treatment compound analysis profile to assist in determination of a disease state of the wound bed after the treatment.

4. The method of claim 3, wherein providing a diagnosis of the wound tissue based on the comparison of compound analysis profiles further comprises:
comparing the post-treatment compound analysis profile with the fragmented wound tissue compound analysis profile wherein each comparison is at a plurality of wound bed locations to determine the change in disease state of the wound bed over the plurality of wound bed locations.

5. The method of claim 1, wherein providing a diagnosis of the wound tissue based on the comparison of compound analysis profiles further comprises:
comparing the fragmented wound tissue compound analysis profile for a plurality of locations on the wound with the database of known compound analysis profiles wherein each comparison determines the disease state of the wound tissue over the plurality of wound locations in situ.

6. The method of claim 1, further comprising:
gathering a molecular gaseous sample emitted from a location of healthy tissue of a same tissue type as the wound tissue for a control compound analysis;
storing a control compound analysis profile in the database of known compound analysis profiles.

7. The method of claim 6, wherein providing a diagnosis of the wound tissue based on the comparison of compound analysis profiles further comprises:
contrasting the fragmented wound tissue compound analysis profile with the control compound analysis profile to determine the disease state of the wound tissue.

8. The method of claim 1, further comprising:
gathering a molecular gaseous sample emitted from a location on the wound bed before removal of the wound tissue for a pre-treatment compound analysis;
storing a pre-treatment compound analysis profile in the database of known compound analysis profiles; and
comparing the pre-treatment compound analysis profile with the known compound analysis profiles to determine the type of pathogens present in the wound bed.

9. The method of claim 1, further comprising:
gathering a molecular gaseous sample emitted from a location on the wound bed before removal of the wound tissue for a pre-treatment compound analysis;
storing a pre-treatment compound analysis profile in the database of known compound analysis profiles; and
comparing the pre-treatment compound analysis profile with the known compound analysis profiles to determine the type of biofilms in the wound bed.

10. The method of claim 1, wherein providing a diagnosis of the wound tissue based on the comparison of compound analysis profiles further comprises:
comparing the fragmented wound tissue compound analysis profile with the known compound analysis profiles to determine the type of tissue removed by treatment.

11. The method of claim 1, wherein providing a diagnosis of the wound tissue based on the comparison of compound analysis profiles further comprises:
comparing the fragmented wound tissue compound analysis profile with the known compound analysis profiles to determine the type of pathogens present in situ.

12. The method of claim 11, wherein providing a diagnosis of the wound tissue based on the comparison of compound analysis profiles further comprises:
determining the level of pathogen infection present in the wound tissue in situ based on the fragmented wound tissue compound analysis profile.

13. The method of claim 1, wherein providing a diagnosis of the wound tissue based on the comparison of compound analysis profiles further comprises:
comparing the fragmented wound tissue compound analysis profile with the known compound analysis profiles to determine the type of biofilms present in situ.

14. The method of claim 13, wherein providing a diagnosis of the wound tissue based on the comparison of compound analysis profiles further comprises:
determining the level of pathogen infection present in the biofilm in situ based on the fragmented wound tissue compound analysis profile.

15. A method comprising:
gathering a molecular gaseous sample emitted from a location on a wound bed after electrosurgical treatment of the wound tissue location;
analyzing the molecular gaseous sample emitted from a location on a wound bed after electrosurgical treatment to generate a post-treatment compound analysis profile;
comparing the post-treatment compound analysis profile with a database of known compound analysis profiles; and
providing a diagnosis of the wound bed location based on a comparison of the compound analysis profiles.

16. The method of claim 15, wherein the diagnosis is provided to assist in determination of a disease state of the wound bed location after the electrosurgical treatment.

17. The method of claim 15, further comprising:
gathering a molecular gaseous sample emitted from the location on the wound bed before electrosurgical treatment of the wound tissue;
analyzing the molecular gaseous sample emitted from a location on a wound bed before electrosurgical treatment to generate a pre-treatment compound analysis profile;
storing a pre-treatment compound analysis profile in the database of known compound analysis profiles; and
comparing the pre-treatment compound analysis profile with the known compound analysis profiles to assist in determination of the disease state in the wound bed location before electrosurgical treatment.

18. The method of claim 15, wherein providing a diagnosis of the wound bed location based on the comparison of compound analysis profiles further comprises:
comparing the post-treatment compound analysis profile with the pre-treatment compound analysis profile at a plurality of wound bed locations to determine the change in disease state of the wound bed over the plurality of wound bed locations.

19. The method of claim 15, wherein providing a diagnosis of the wound bed location based on the comparison of compound analysis profiles further comprises:
comparing the post-treatment compound analysis profile with the known compound analysis profiles to determine the type of tissue remaining in the wound bed location after electrosurgical treatment.

20. The method of claim 15, wherein providing a diagnosis of the wound bed location based on the comparison of compound analysis profiles further comprises:
comparing the fragmented wound tissue compound analysis profile with the known compound analysis profiles to determine the type of pathogens remaining in the wound bed location after electrosurgical treatment.

21. The method of claim 20, wherein providing a diagnosis of the wound bed location based on the comparison of compound analysis profiles further comprises:
determining the level of pathogen infection present in the wound tissue location after treatment based on the post-treatment compound analysis profile.

22. The method of claim 15, wherein providing a diagnosis of the wound bed location based on the comparison of compound analysis profiles further comprises:
comparing the post-treatment compound analysis profile with the known compound analysis profiles to determine the type of biofilm remaining in the wound bed location after treatment.

23. The method of claim 22, wherein providing a diagnosis of the wound bed location based on the comparison of compound analysis profiles further comprises:
determining the level of pathogen infection present in the biofilm after treatment based on the post-treatment compound analysis profile.

24. A system for electrosurgically treating tissue comprising:
an electrosurgical treatment mechanism to provide electrosurgical treatment to a target tissue wherein the target tissue is fragmented;
a sampling aperture to gather a molecular gaseous by-product sample of tissue fragmentation;
a sensor in fluid communication with the sampling aperture to detect compounds from a molecular gaseous by-product sample of tissue fragmentation;
a processor to determine a fragmented target tissue compound analysis profile; and
the processor comparing the fragmented target tissue compound profile with a database of known compound analysis profiles resulting from the target tissue fragmentation.

25. The system of claim 24, wherein the electrosurgical treatment mechanism further comprises:
an electrosurgical probe having a distal end including at least one active electrode disposed near the distal end,
wherein the electrosurgical probe fragments tissue via plasma-based volumetric dissociation.

26. The system of claim 24, further comprising:
the sampling aperture to gather a molecular gaseous sample emitted from a location on the remaining target tissue bed for a post-treatment compound analysis after electrosurgical treatment of the target tissue;
the processor to compare a post-treatment compound analysis profile with a database of known compound analysis profiles; and
the processor to provide a post-treatment diagnosis of the remaining target tissue at the target bed location based on the comparison of the post-treatment compound analysis profile to assist in determination of a disease state of the target tissue bed after the treatment.

27. The system of claim 26, further comprising:
a treatment site navigation detector to determine target tissue locations in a target tissue bed;
the processor to compare the post-treatment compound analysis profile with the fragmented target tissue compound analysis profile wherein each comparison is at a plurality of target bed locations to determine the change in disease state of the target tissue bed over the plurality of target bed locations.

28. The method of claim 24, further comprising:
a treatment site navigation detector to determine target tissue locations in a target tissue bed; and
the processor to compare the fragmented target tissue compound analysis profile for a plurality of locations on the target tissue with the database of known compound analysis profiles resulting from the target tissue fragmentation wherein each comparison determines the disease state of the target tissue over the plurality of target tissue bed locations in situ.

29. The system of claim 24, further comprising:
the sampling aperture to gather a molecular gaseous sample emitted from a location of healthy tissue of a same tissue type as the target tissue; and
the processor determining a control compound analysis profile of the healthy tissue for storage in the database of known compound analysis profiles.

30. The system of claim 29, further comprising:
the processor to provide a target tissue diagnosis by contrasting the fragmented target tissue compound analysis profile with the control compound analysis profile to determine the disease state of the target tissue.

31. The system of claim 24, further comprising:
the sampling aperture to gather a molecular gaseous sample emitted from a location on the target tissue bed before electrosurgical removal of the target tissue for a pre-treatment compound analysis by the sensor;
the processor to store a pre-treatment compound analysis profile in the database of known compound analysis profiles; and
the processor to compare the pre-treatment compound analysis profile with the known compound analysis profiles resulting from the target tissue fragmentation to determine the type of pathogens present in the target tissue bed.

32. The system of claim 24, further comprising:
the sampling aperture to gather a molecular gaseous sample emitted from a location on the target tissue bed before electrosurgical removal of the target tissue for a pre-treatment compound analysis;
the processor to store a pre-treatment compound analysis profile in the database of known compound analysis profiles resulting from the target tissue fragmentation; and
the processor to compare the pre-treatment compound analysis profile with the known compound analysis profiles to determine the type of biofilms in the target tissue bed.

33. The system of claim 24, further comprising;
the processor providing a diagnosis of the target tissue based on the comparison the fragmented target tissue compound analysis profile with database of known compound analysis profiles resulting from the target tissue fragmentation to determine the type of tissue removed by treatment.

34. The system of claim 24, further comprising;
the processor providing a diagnosis of the target tissue based on the comparison the fragmented target tissue compound analysis profile with database of known compound analysis profiles resulting from the target tissue fragmentation to determine the type of pathogens present in situ.

35. The system of claim 34, further comprising:
the processor to further determine the level of pathogen infection present in the target tissue in situ based on the detected compound intensity levels in the fragmented target tissue compound analysis profile.

36. The system of claim 24, further comprising;
the processor providing a diagnosis of the target tissue based on the comparison the fragmented target tissue compound analysis profile with database of known compound analysis profiles resulting from the target tissue fragmentation to determine the type of biofilms present in situ.

37. The system of claim 36, further comprising:
the processor to further determine the level of pathogen infection present in the biofilm in situ based on the detected compound intensity levels in the fragmented target tissue compound analysis profile.

38. A system for diagnosing treated tissue comprising:
a sampling aperture to gather a molecular gaseous sample of target tissue fragmented by electrosurgical or non-electrosurgical treatment;
a sensor in fluid communication with the sampling aperture to detect compounds from a sample of the molecular gaseous by-product of target tissue fragmentation;
a processor to determine a fragmented target tissue compound analysis profile; and
the processor to compare the compound profile with a database of known compound analysis profiles resulting from the target tissue fragmentation.

39. The system of claim 38, wherein the target tissue fragmentation further comprises:
plasma-based volumetric dissociation of the target tissue.

40. The system of claim 38, further comprising:
the sampling aperture to gather a molecular gaseous sample emitted from a location on the remaining target tissue bed for a post-treatment compound analysis after electrosurgical or non-electrosurgical treatment of the target tissue;
the processor to compare a post-treatment compound analysis profile with a database of known compound analysis profiles; and
the processor to provide a post-treatment diagnosis of the remaining target tissue at the target bed location based on the comparison of the post-treatment compound analysis profile to assist in determination of a disease state of the target tissue bed after the treatment.

41. The system of claim 40, further comprising:
a treatment site navigation detector to determine target tissue locations in a target tissue bed;
the processor to compare the post-treatment compound analysis profile with the fragmented target tissue compound analysis profile wherein each comparison is at a plurality of target bed locations to determine the change in disease state of the target tissue bed over the plurality of target bed locations.

42. The method of claim 38, further comprising:
a treatment site navigation detector to determine target tissue locations in a target tissue bed; and
the processor to compare the fragmented target tissue compound analysis profile for a plurality of locations on the target tissue with the database of known compound analysis profiles resulting from the target tissue fragmentation wherein each comparison determines the disease state of the target tissue over the plurality of target tissue bed locations in situ.

43. The system of claim 38, further comprising:
the sampling aperture to gather a molecular gaseous sample emitted from a location of healthy tissue of a same tissue type as the target tissue; and
the processor determining a control compound analysis profile of the healthy tissue for storage in the database of known compound analysis profiles.

44. The system of claim 43, further comprising:
the processor to provide a target tissue diagnosis by contrasting the fragmented target tissue compound analysis profile with the control compound analysis profile to determine the disease state of the target tissue.

45. The system of claim 38, further comprising:
the sampling aperture to gather a molecular gaseous sample emitted from a location on the target tissue bed before removal of the target tissue for a pre-treatment compound analysis by the sensor;
the processor to store a pre-treatment compound analysis profile in the database of known compound analysis profiles; and
the processor to compare the pre-treatment compound analysis profile with the known compound analysis profiles resulting from the target tissue fragmentation to determine the type of pathogens present in the target tissue bed.

46. The system of claim 38, further comprising:
the sampling aperture to gather a molecular gaseous sample emitted from a location on the target tissue bed before removal of the target tissue for a pre-treatment compound analysis;
the processor to store a pre-treatment compound analysis profile in the database of known compound analysis profiles resulting from the target tissue fragmentation; and
the processor to compare the pre-treatment compound analysis profile with the known compound analysis profiles to determine the type of biofilms in the target tissue bed.

47. The system of claim 38, further comprising;
the processor providing a diagnosis of the target tissue based on the comparison the fragmented target tissue compound analysis profile with database of known compound analysis profiles resulting from the target tissue fragmentation to determine the type of tissue removed by treatment.

48. The system of claim 38, further comprising;
the processor providing a diagnosis of the target tissue based on the comparison of the fragmented target tissue compound analysis profile with database of known compound analysis profiles resulting from the target tissue fragmentation to determine the type of pathogens present in situ.

49. The system of claim 48, further comprising:
the processor to further determine the level of pathogen infection present in the target tissue in situ based on the detected compound intensity levels in the fragmented target tissue compound analysis profile.

50. The system of claim 38, further comprising;
the processor providing a diagnosis of the target tissue based on the comparison the fragmented target tissue compound analysis profile with database of known compound analysis profiles resulting from the target tissue fragmentation to determine the type of biofilms present in situ.

51. The system of claim 50, further comprising:
the processor to further determine the level of pathogen infection present in the biofilm in situ based on the detected compound intensity levels in the fragmented target tissue compound analysis profile.

52. A system for diagnosing electrosurgically treated tissue comprising:
a sampling aperture to gather a molecular gaseous by-product sample of target tissue fragmented by electrosurgical treatment;
a sensor in fluid communication with the sampling aperture to detect compounds from a sample of the molecular gaseous by-product of tissue fragmentation;
a processor to determine a fragmented target tissue compound analysis profile; and
the processor to subtract out one or more data signatures specific to electrosurgical treatment of the target tissue from the fragmented target tissue compound analysis profile resulting in a diagnostic compound analysis profile;
the processor to compare the diagnostic compound profile with a database of known compound analysis profiles.

53. The system of claim 52, wherein the target tissue fragmentation further comprises:
plasma-based volumetric dissociation of the target tissue.

54. The system of claim 52, further comprising:
the sampling aperture to gather a molecular gaseous sample emitted from a location on the remaining target tissue bed for a post-treatment compound analysis after electrosurgical treatment of the target tissue;
the processor to compare a post-treatment compound analysis profile with a database of known compound analysis profiles; and
the processor to provide a post-treatment diagnosis of the remaining target tissue at the target bed location based on the comparison of the post-treatment compound analysis profile to assist in determination of a disease state of the target tissue bed after the treatment.

55. The system of claim 54, further comprising:
a treatment site navigation detector to determine target tissue locations in a target tissue bed;
the processor to compare the post-treatment compound analysis profile with the fragmented target tissue compound analysis profile wherein each comparison is at a plurality of target bed locations to determine the change in disease state of the target tissue bed over the plurality of target bed locations.

56. The method of claim 52, further comprising:
a treatment site navigation detector to determine target tissue locations in a target tissue bed; and
the processor to compare the fragmented target tissue compound analysis profile for a plurality of locations on the target tissue with the database of known compound analysis profiles resulting from the target tissue fragmentation wherein each comparison determines the disease state of the target tissue over the plurality of target tissue bed locations in situ.

57. The system of claim 52, further comprising:
the sampling aperture to gather a molecular gaseous sample emitted from a location of healthy tissue of a same tissue type as the target tissue; and
the processor determining a control compound analysis profile of the healthy tissue for storage in the database of known compound analysis profiles.

58. The system of claim 57, further comprising:
the processor to provide a target tissue diagnosis by contrasting the fragmented target tissue compound analysis profile with the control compound analysis profile to determine the disease state of the target tissue.

59. The system of claim 52, further comprising:
the sampling aperture to gather a molecular gaseous sample emitted from a location on the target tissue bed before electrosurgical removal of the target tissue for a pre-treatment compound analysis by the sensor;
the processor to store a pre-treatment compound analysis profile in the database of known compound analysis profiles; and
the processor to compare the pre-treatment compound analysis profile with the known compound analysis profiles resulting from the target tissue fragmentation to determine the type of pathogens present in the target tissue bed.

60. The system of claim 52, further comprising:
the sampling aperture to gather a molecular gaseous sample emitted from a location on the target tissue bed before electrosurgical removal of the target tissue for a pre-treatment compound analysis;
the processor to store a pre-treatment compound analysis profile in the database of known compound analysis profiles resulting from the target tissue fragmentation; and
the processor to compare the pre-treatment compound analysis profile with the known compound analysis profiles to determine the type of biofilms in the target tissue bed.

61. The system of claim 52, further comprising;
the processor providing a diagnosis of the target tissue based on the comparison the fragmented target tissue compound analysis profile with database of known compound analysis profiles resulting from the target tissue fragmentation to determine the type of tissue removed by treatment.

62. The system of claim 52, further comprising;
the processor providing a diagnosis of the target tissue based on the comparison the fragmented target tissue compound analysis profile with database of known compound analysis profiles resulting from the target tissue fragmentation to determine the type of pathogens present in situ.

63. The system of claim 62, further comprising:
the processor to further determine the level of pathogen infection present in the target tissue in situ based on the detected compound intensity levels in the fragmented target tissue compound analysis profile.

64. The system of claim 52, further comprising;
the processor providing a diagnosis of the target tissue based on the comparison the fragmented target tissue compound analysis profile with database of known compound analysis profiles resulting from the target tissue fragmentation to determine the type of biofilms present in situ.

65. The system of claim 64, further comprising:
the processor to further determine the level of pathogen infection present in the biofilm in situ based on the detected compound intensity levels in the fragmented target tissue compound analysis profile.

66. A system for diagnosing electrosurgically treated tissue comprising:
a sampling aperture to gather a molecular gaseous sample emitted from a location on a target tissue bed for a compound analysis after electrosurgical treatment of a target tissue location;
a sensor in fluid communication with the sampling aperture to detect compounds from the molecular gaseous sample emitted from a location on the target tissue bed; and
a processor to compare a post-treatment compound analysis profile of the molecular gaseous sample emitted from the target tissue bed with a database of known compound analysis profiles resulting from post-electrosurgical treatment,
wherein the comparison is provided to assist in determination of a disease state of the target tissue bed location after the electrosurgical treatment.

67. The system of claim 66, further comprising:
the sampling aperture to gather a molecular gaseous sample emitted from the location on the target tissue bed before removal of the target tissue for a pre-treatment compound analysis; and
the processor to determine a pre-treatment compound analysis profile; and
the processor to compare the post-treatment compound analysis profile with the pre-treatment compound analysis profile to determine the change in disease state of the target tissue bed.

68. The system of claim 68, further comprising:
a treatment site navigation detector to determine target tissue locations in a target tissue bed;
the processor to compare the post-treatment compound analysis profile with the pre-treatment compound analysis profile at a plurality of target tissue bed locations to determine the change in disease state of the target tissue bed over the plurality of target bed locations.

69. The system of claim 66, further comprising:
the processor to compare the post-treatment compound analysis profile with the known compound analysis profiles resulting from post-electrosurgical treatment to determine the type of tissue remaining in the target tissue bed location after electrosurgical treatment.

70. The system of claim 66, further comprising:
the processor to compare the post-treatment compound analysis profile with the known compound analysis profiles resulting from post-electrosurgical treatment to determine the type of pathogens remaining in the target tissue bed location after electrosurgical treatment.

71. The system of claim 70, further comprising:
the processor to determine the level of pathogen infection present in the target tissue bed location after treatment based on the post-treatment compound analysis profile.

72. The system of claim 66, further comprising:
the processor to compare the post-treatment compound analysis profile with the known compound analysis profiles resulting from post-electrosurgical treatment to determine the type of biofilm remaining in the target tissue bed location after treatment.

73. The system of claim 72, further comprising:
the processor to determine the level of pathogen infection present in the biofilm after treatment based on the post-treatment compound analysis profile.

74. A method comprising:
segmenting a wound bed into wound bed location zones identified by a treatment site navigation detector;
gathering molecular gaseous samples emitted from the plurality of wound bed locations;
analyzing the molecular gaseous samples emitted from a plurality of wound bed location zones on a wound bed to generate a plurality compound analysis profiles for the plurality of wound bed location zones;
providing diagnoses for the plurality wound bed location zones; and
mapping the diagnoses for the plurality wound bed location zones, wherein the diagnoses mapping is provided to assist in determination of a disease state of the wound bed for treatment.

75. The method of claim 74, wherein the segmenting the wound bed further comprises:
segmenting the wound bed into a grid of wound bed location zones.

76. The method of claim 74, wherein the diagnosis mapping further comprises:
a graphical representation of the wound bed location zones with associated diagnoses to assist in navigation of treatment of zones of the wound bed.

77. The method of claim 76, wherein the diagnosis mapping further comprises:
a tracking identifier of an electrosurgical treatment mechanism showing the location of the electrosurgical treatment mechanism on the graphical representation of the wound bed location zones.

78. The method of claim 74, wherein the treatment site navigation detector is an optical treatment site navigation system.

79. The method of claim 74, wherein the treatment site navigation detector is an electromagnetic treatment site navigation system.
